(19) United States
(12) Patent Application Publication
(10) Pub. No.: US 2024/0058419 A1
(43) Pub. Date: Feb. 22, 2024

(54) METHOD FOR TREATING OPTIC NEUROPATHY

(71) Applicant: BUDDHIST TZU CHI MEDICAL FOUNDATION, Hualien (TW)

(72) Inventors: Rong-Kung TSAI, Hualien (TW); Yao-Tseng WEN, Hualien (TW)

(21) Appl. No.: 17/821,416

(22) Filed: Aug. 22, 2022

(51) Int. Cl. A61K 38/19; A61K 47/60; A61P 27/02

(52) U.S. Cl. CPC A61K 38/193; A61K 47/60; A61P 27/02

(57) ABSTRACT

Provided is a pharmaceutical composition for treating optic neuropathy, including a long-acting granulocyte-colony stimulating factor (G-CSF) and a pharmaceutically acceptable excipient thereof. Also provided is a method for treating optic neuropathy in a subject in need thereof, including administering to the subject with the long-acting G-CSF.

### BACKGROUND

### 1. Technical Field

The present disclosure relates generally to treatment of optic neuropathy. In particular, the present disclosure relates to a method for treating optic neuropathy with a long-acting granulocyte-colony stimulating factor (G-CSF).

### 2. Description of Related Art

The optic nerve (ON) contains axons of nerve cells that emerge from the retina, leave the eye at the optic disc, and go to the visual cortex of the brain where input from the eye is processed into vision. Optic neuropathy refers to damage to the optic nerve due to any cause. Damage and death of the nerve cells, leading to characteristic features of optic neuropathy. The main symptom is loss of vision, with colors appearing subtly washed out in the affected eye.

Traumatic optic neuropathy (TON) indicates to insults to the optic nerve secondary to trauma. It can be identified according to the site of injury (e.g., ON head, intraorbital, intracanalicular, or intracranial injury) or depending on the type of injury (e.g., direct or indirect injury)^{[1,2]}. Direct TON exhibits a severe anatomical disruption to the ON, for example, from a projectile penetrating the orbit at high velocity, or as a result of ON avulsion^{[3]}. The transmission of forces to the ON from a distant site can cause indirect TON, without any obvious damage. After trauma, there is an instant decreasing axons of retinal ganglion cells (RGCs), which is an irreversible action that leads to neuronal loss^{[4]}. ON swelling within the tight confines of the optic canal secondary to direct mechanical trauma and vascular ischemia can be observed^{[5]}. The following syndrome further damages the already compromised blood supply to surviving retinal ganglion cells, developing toward apoptotic cell death^{[6]}.

TON is a cause of visual loss following blunt or penetrating head trauma with an incidence of 0.7% to 2.5%. A national epidemiological survey of TON in the United Kingdom found a minimum prevalence in the general population of one in 1,000,000. The enormous majority of affected patients are young adult males (79% to 85%). Motor vehicle and bicycle accidents (49%), falls (27%), and assaults (13%) are the most common causes of TON^{[7,8]}. In the pediatric population, secondary to falls (50%) and road traffic accidents (40%) are the majority of TON cases^{[9]}.

Since the exact pathophysiology of TON is unclear, its management has remained controversialm. Three common managements are observation, corticosteroid, and/or optic canal decompression surgery. Corticosteroids and optic canal decompression surgery have not shown any significantly better visual outcome than observation in the literature^{[11]}. However, one meta-analysis concluded that treatment with corticosteroids, optic canal decompression surgery, or both is better than no treatment^{[12]}. The results of these medical and surgical interventions have shown to be uncertain with possible serious side effects or complications^{[13-15]}. There has been no study which could validate a particular approach to the management of TON.

Hence, there is no effective treatment recommended for patients with TON, and development of alternative therapy for patients with TON is urgent and necessary.

### SUMMARY

In view of the foregoing, the present disclosure provides a long-acting granulocyte-colony stimulating factor (G-CSF) that is capable of providing neuroprotection to optic nerve, thereby preventing, arresting progression of or ameliorating vision loss associated with optic neuropathy. In at least one aspect, the present disclosure provides a pharmaceutical composition for treating optic neuropathy in a subject in need thereof, wherein the pharmaceutical composition comprises an effective amount of a long-acting G-CSF and a pharmaceutically acceptable excipient thereof.

In at least one embodiment of the present disclosure, the optic neuropathy may be ischemic optic neuropathy, optic neuritis, compressive optic neuropathy, infiltrative optic neuropathy, traumatic optic neuropathy, mitochondrial optic neuropathy, nutritional optic neuropathy, toxic optic neuropathy, radiation optic neuropathy, hereditary optic neuropathy, or any combination thereof.

In at least one embodiment of the present disclosure, the long-acting G-CSF is at least one selected from the group consisting of a recombinant G-CSF, a conjugated G-CSF, a G-CSF fusion protein, and any combination thereof. In some embodiments, the conjugated G-CSF is a non-immunogenic hydrophilic polymer linked to G-CSF. In some embodiments, the non-immunogenic hydrophilic polymer is covalently linked to the G-CSF. In some embodiments, the G-CSF fusion protein comprises G-CSF fused to a protein selected from the group consisting of albumin and an immunoglobulin fragment of IgG. In some embodiments, the G-CSF is fused to the immunoglobulin Fc fragment of the IgG.

In at least one embodiment of the present disclosure, the non-immunogenic hydrophilic polymer is at least one selected from the group consisting of polyethylene glycol (PEG), polyoxypropylene, polyoxyethylene-polyoxypropylene block copolymer, polyvinylpyrrolidone, polyacyloylmorpholine, polysaccharide, aminocarbamyl polyethylene glycol, and any combination thereof. In some embodiments, the long-acting G-CSF is a polyethylated G-CSF (PEG-GCSF).

In at least one embodiment of the present disclosure, the pharmaceutical composition is administered orally, intravitreally, intraperitoneally, intravenously, intradermally, intramuscularly, subcutaneously, or transdermally. In some embodiments, the administering route of the pharmaceutical composition is intravitreal injection.

In at least one embodiment of the present disclosure, the effective amount of the long-acting G-CSF for administration is in a range of from about 1 μg to about 2 such as from about 1.1 μg to about 1.9 from about 1.2 μg to about 1.8 from about 1.3 μg to about 1.7 from about 1.4 μg to about 1.6 and from about 1 μg to about 1.5 μg. In some embodiments, the effective amount of the long-acting G-CSF for administration is about 1 μg, about 1.1 μg, about 1.2 μg, about 1.3 μg, about 1.4 μg, about 1.5 μg, about 1.6 μg, about 1.7 μg, about 1.8 μg, about 1.9 μg, about 2.0 μg. In some embodiments, the effective amount of the long-acting G-CSF administered to humans is in a range of from about 1 μg to about 2 μg.

In at least one embodiment of the present disclosure, the effective amount of the long-acting G-CSF for administration is in a range of from about 10 ng to about 100 ng, such as from about 15 ng to about 95 ng, from about 20 ng to about 80 ng, from about 25 ng to about 75 ng, from about 30 ng to about 60 ng, and from about 35 ng to about 55 ng. In some embodiments, the effective amount of the long-acting G-CSF for administration is about 10 ng, about 15 ng, about 20 ng, about 25 ng, about 26 ng, about 27 ng, about 28 ng, about 29 ng, about 30 ng, about 31 ng, about 32 ng, about 33 ng, about 34 ng, about 35 ng, about 36 ng, about 37 ng, about 38 ng, about 39 ng, about 40 ng, about 45 ng, about 50 ng, about 60 ng, about 70 ng, about 80 ng, about 90 ng, or about 100 ng. In some embodiments, the effective amount of the long-acting G-CSF administered to rats is in a range of from about 1 μg to about 2 μg.

In at least one embodiment of the present disclosure, the pharmaceutical composition is administered to the subject 1 to 4 times during the treatment, such as 2 times during the treatment and 3 times during the treatment. In some embodiments, the pharmaceutical composition is administered to the subject only one time during the treatment. In some embodiments, the method of the present disclosure comprises administering one single shot of the pharmaceutical composition to the subject within, e.g., one month, after the optic neuropathy occurs.

Granulocyte-colony stimulating factor (G-CSF) has neuroprotective effects in the optic nerve crush (ONC) model and in a rat model of anterior ischemic optic neuropathy (rAION) via the dual actions of anti-apoptosis of RGCs and anti-inflammation on the ON. In addition, G-CSF and its receptors are endogenous ligands in the central nervous system (CNS) and retinal neurons. In some embodiments, treatment with G-CSF can protect RGCs from death via the autocrine protective mechanisms to activate PI3K/AKT pro-surviving signaling in the rat model of traumatic ON injury. However, some treatments with G-CSF is a subcutaneous injection of G-CSF once daily for 5 days, which dramatically induces side effects of leukocytosis. Also, some treatments with G-CSF by intravitreal injection requires repeat injections, which adversely increases inflammation and infection rates. In at least one embodiment of the present disclosure, it shows the effects of the administration of the long-acting G-CSF in the treatment of optic neuropathy without adverse effects. For instance, single shot of intravitreal injection with PEG-GCSF can exhibit excellent neuroprotective effects in traumatic optic neuropathy.

These and other aspects will become apparent from the following descriptions of the embodiments taken in conjunction with the drawings, although variations and modifications therein may be affected without departing from the scope of the present disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure can be more fully understood by reading the following descriptions of the embodiments, with reference made to the accompanying drawings.

FIGS. 1A and 1B show the effects of PEG-GCSF on flash visual evoked potentials (FVEPs) in normal rats. The amplitudes of the P1-N2 are expressed as mean±SD in each group.

FIG. 2 shows the effects of PEG-GCSF on leukocytosis in optic nerve crush (ONC) model, in which the white blood cell (WBC) count is determined on day 7 post intravitreal injection with PEG-GCSF (PG). Data are expressed as mean±SD in each group.

FIGS. 3A and 3B show the effects of PEG-GCSF on flash visual evoked potentials (FVEPs) in optic nerve crush (ONC) model. The amplitudes of the P1-N2 are expressed as mean±SD in each group. Asterisk (*) indicates p<0.05 by using Mann-Whitney U test. ONC: optic nerve crush; PBS: phosphate buffered saline.

FIG. 4 shows the representative of flat-mounted central retinas and the morphometry of retinal ganglion cells (RGCs) in each group by FluoroGold retrograde labeling. Data are expressed as mean±SD in each group. Asterisk (*) indicates p<0.05 by using Mann-Whitney U test. ONC: optic nerve crush; PBS: phosphate buffered saline.

FIGS. 5A to 5C show the evaluation of PEG-GCSF on inflammatory infiltration and microglia activation in optic nerve crush (ONC) model. FIG. 5A shows that ED1 and ionized calcium binding adaptor molecule 1 (IBA1) staining in the longitudinal sections of ON. The ED1 positive cells are labeled in red, and IBA1 positive cells are labeled in green. The nuclei of ON are stained in blue with 4′,6-diamidino-2-phenylindole (DAPI).

FIGS. 5B and 5C show the quantification of ED1 positive (EDI+) cell and IBA1 positive (IBA1+) cell per high power field (HPF). Data are expressed as mean±SD in each group. Asterisk (*) indicates p<0.05 by using Mann-Whitney U test. ONC: optic nerve crush; PBS: phosphate buffered saline.

FIG. 6 shows the effect of PEG-GCSF on RGC apoptosis in optic nerve crush (ONC) model, in which RGC death in the RGC layer is analyzed by the TUNEL assay. The apoptotic cells (TUNEL positive cells) in green are stained with TUNEL staining, and the nuclei of RGCs in blue are stained with DAPI staining. The quantification of TUNEL positive (TUNEL+) cell per high power field (HPF) is illustrated. Data are expressed as mean±SD in each group. Asterisk (*) indicates p<0.05 by using Mann-Whitney U test. ONC: optic nerve crush; PBS: phosphate buffered saline; GCL: ganglion cell layer; INL: inner nuclear layer.

FIG. 7A illustrates the logMAR BCVA before intervention, on day 1, day 7, day 30, and day 90 with the Neulasta treatment of the participants.

FIG. 7B illustrates the proportion of stable outcome, improved outcome, and reduced outcome with the Neulasta treatment of the participants.

FIG. 8 shows the logMAR BCVA before intervention, on day 1, day 7, day 30, and day 90 with the Neulasta treatment of the participants.

FIG. 9 shows the intraocular pressure before intervention, on day 30, and day 90 of the participants.

FIG. 10 shows the visual field MD(-dB) before intervention, on day 30 and day 90 of the participants.

FIG. 11 shows the WBC count before intervention, on day 1, day 7, day 30, and day 90 of the participants.

FIG. 12 shows the initial BCVA, initial visual field (db), 90 days post treatment BCVA, and 90 days post treatment visual field (dB) of the participants.

### DETAILED DESCRIPTION

The following examples are used for illustrating the present disclosure. A person skilled in the art can easily conceive the other advantages and effects of the present disclosure, based on the disclosure of the specification. The present disclosure can also be implemented or applied as described in different examples. It is possible to modify or alter the following examples for carrying out this disclosure without contravening its scope, for different aspects and applications.

It is further noted that, as used in this disclosure, the singular forms “a,” “an,” and “the” include plural referents, unless expressly and unequivocally limited to one referent. The term “or” is used interchangeably with the term “and/or,” unless the context clearly indicates otherwise.

As used herein, the term “comprising” or “comprises” is used in reference to compositions, methods, and respective component(s) thereof, which are included in the present disclosure, yet open to the inclusion of unspecified elements.

The present disclosure is directed to a method for treating optic neuropathy in a subject in need thereof. In the present disclosure, it is surprisingly found that intravitreal injection with a long-acting G-CSF, such as PEG-GCSF, may preserve visual function and RGC density after the optic neuropathy occurs. It is also found that intravitreal injection with the long-acting G-CSF may inhibit macrophage infiltration into ON and RGC apoptosis. Accordingly, the present disclosure provides a method for treating optic neuropathy by administering an effective amount of a long-acting G-CSF to a subject in need thereof.

In some embodiments, the optic neuropathy may be selected from such conditions as, but not limited to: traumatic neuropathy (that may result from any type of trauma to the optic nerve), ischemic neuropathy (such as nonarteritic anterior ischemic optic neuropathy (NAION)), anterior ischemic optic neuropathy (AION), and posterior ischemic optic neuropathy), radiation optic neuropathy (RON), optic neuritis, compressive optic neuropathy, infiltrative optic neuropathy, mitochondrial optic neuropathy, nutritional optic neuropathy, toxic optic neuropathy, hereditary optic neuropathy, and the like.

As used herein, the term “long-acting G-CSF” is intended to refer to a protein construct in which the physiologically active G-CSF has a prolonged duration of action compared to G-CSF in its natural form. The term “long-acting,” as used herein, refers to a prolonged duration of action compared to that of a natural form.

For use in the present disclosure, the G-CSF has an amino acid sequence of human G-CSF or closely related analogues. The G-CSF useful in the present disclosure may be a naturally occurring protein or a recombinant protein. Also, the G-CSF may be a mutant one that has undergone the addition, deletion or insertion of amino acids, provided that the mutation does not have a significant influence on the original biological activity thereof.

In at least one embodiment of the present disclosure, the long-acting G-CSF may be a recombinant G-CSF, a conjugated G-CSF, or a G-CSF fusion protein. The term “conjugated G-CSF” or “G-CSF conjugate” refers to a construct in which G-CSF and one or more non-immunogenic hydrophilic polymers are covalently linked. The term “G-CSF fusion protein” refers to a construct, in which G-CSF and one or more proteins or a fragment, motif or domain thereof, e.g., albumin and an immunoglobulin Fc fragment of IgG, are fused by using a recombinant technique.

In at least one embodiment of the present disclosure, the long-acting G-CSF may be prepared by linking the G-CSF and polyethylene glycol together, so as to form a polyethylated G-CSF (PEG-GCSF).

As used herein, the term “administering” or “administration” refers to the placement of an effective agent (e.g., the long-acting G-CSF) into a subject by a method or route which results in at least partial localization of the effective agent at a desired site such that a desired effect is produced. The effective agent described herein can be administered by any appropriate route known in the art including, but not limited to, oral or parenteral routes, such as intravitreal, intraperitoneal, intravenous, intradermal, intramuscular, subcutaneous, or transdermal routes.

In at least one embodiment of the present disclosure, the long-acting G-CSF may be formulated into a pharmaceutical composition for administration. The pharmaceutical composition comprises, e.g., the above long-acting G-CSF as an effective agent in an effective amount and a pharmaceutically acceptable vehicle thereof.

As used herein, the term “pharmaceutically acceptable vehicle” refers to a pharmaceutically acceptable material, composition, or carrier, such as diluents, disintegrating agents, binders, lubricants, glidants, and surfactants, which does not abrogate the biological activity or properties of the effective agent, and is relatively non-toxic; that is, the material may be administered to a subject without causing an undesirable biological effect or interacting in a deleterious manner with any of the components of the pharmaceutical composition in which it is contained.

As used herein, the phrase “an effective amount” refers to the amount of an effective agent (e.g., the long-acting G-CSF) that is required to confer a desired therapeutic effect (e.g., preserving visual function) on the treated subject. Effective doses will vary, as recognized by one of ordinary skill in the art, depending on routes of administration, excipient usage, the possibility of co-usage with other therapeutic treatment, and the condition to be treated.

As used herein, the terms “treat,” “treating,” and “treatment” refers to the use of an effective agent to a subject in need thereof with the purpose to cure, alleviate, relieve, remedy, ameliorate, reduce, or prevent a disease, a symptom thereof, or predispositions towards it.

As used herein, the term “subject” refers to a mammal, such as a human, but can also be other animals, such as a domestic animal (e.g., a dog, a cat, or the like), a farm animal (e.g., a cow, a sheep, a pig, a horse, or the like) or a laboratory animal (e.g., a monkey, a rodent, a murine, a rabbit, a guinea pig, or the like). The term “participant” or “patient” refers to a “subject” who is suspected to be, or afflicted with a disease or condition. In addition, the term “participant,” “patients,” or “subject” may be used interchangeably.

Many examples have been used to illustrate the present disclosure. The examples below should not be taken as a limit to the scope of the present disclosure.

### EXAMPLES

### Materials and Methods

The materials and methods used in the following Examples 1-6 were described in detail below. The materials used in the present disclosure but unannotated herein were commercially available.

### (1) Study Animal

Wistar rats weighing 150 to 180 g (male, 7 to 8 weeks old), purchased from the breeding colony of BioLASCO Co., Taiwan, were used in the study. Animal care and experimental procedures were followed in accordance with the statement of Association for Research in Vision and Ophthalmology (ARVO) for the use of animals in ophthalmic and vision research. The rats were maintained under a controlled 12-h shift of the light-dark cycles and had free access to food and water in a controlled environment with a constant temperature of 23° C. and constant humidity of 55%. All operations were performed with the animals under general anesthesia, which was achieved by intramuscular administration of a mixture of ketamine (100 mg/kg body weight) and xylazine (10 mg/kg body weight; Sigma, St. Louis, MO, USA). The Institutional Animal Care and Use Committee at Hualien Tzu-Chi Hospital (Taiwan) approved all animal experiments.

### (2) Optic Nerve Crush (ONC) Experiments

An ONC injury was induced as the method previously described^{[16]}. Briefly, after typical anesthesia and topical Alcaine eye drop administration, the right ON was uncovered and isolated. The surgery was performed carefully to avoid damaging the small vessels around the ON. A standardized ONC with a vascular clip (60-g microvascular clip, World Precision Instruments, FL, USA) was subsequently applied to the ON at a distance of 2 mm posterior to the globe for 30 seconds (s). After the operation, Tobradex eye ointment (Alcon, Puurs, Belgium) was administered. Lastly, the rats were kept on electric heating pads at 37° C. for recovery. The sham group of rats received a sham operation on right eye that represents ON exposure without the crush operation.

### (3) Study Design

The ON crushed rats were further allocated into 4 groups. The first group was the rats that were intravitreally administrated with single shot (5 μL) of PEG-GCSF (Neulasta, Amgen, Inc.) on day 0 post-ONC (n=36). The second group was the rats that were intravitreally administrated with double shots (5 μL*2) of PEG-GCSF on day 0 and 14 post-ONC (n=36). The third group was the rats that were intravitreally administrated with triple shots (5 μL*3) of PEG-GCSF on day 0, 14, and 28 post-ONC (n=24). The fourth group was the operated rats that were not given any treatment (n=12). Another 12 rats without ONC operation were allocated into the shame group. RGC density was measured by retrograde labeling with FluoroGold, and visual function was assessed by photoptic flash visual-evoked potentials (FVEP) on 2, 4, 6, and 8 weeks post-ONC. Terminal deoxynucleotidyl transferase dUTP nick end-labeling (TUNEL) assays in the RGC layer was also conducted. Extrinsic macrophage (ED1) markers and microglia marker (IBA1) in the ON sections were investigated by immunohistochemistry (IHC).

### (4) Intravitreal Injection of PEG-GCSF

Intravitreal injection of PEG-GCSF (Neulasta, Amgen, Inc.) was performed as described previously^{[17]}. Briefly, rats were anesthetized with an intramuscular injection of a mixture of ketamine-xylazine (40 mg/kg and 4 mg/kg, respectively). A single 3 μL injection of 30 ng of PEG-GCSF was administered intravitreally into the eye of the ON-crushed rat under a microscope to avoid lens injury. The 33-G needles (Hamilton7747-01 with a Gastight syringe, IA2-1701RN 10-1L SYR; Hamilton Co., Hamilton, KS, USA) were used to perform the intravitreal injections. Intraocular pressure (TOP) was measured using a Tono-Pen (Reichert Technologies, Depew, NY, USA) 1 day after the intravitreal injections.

(5) Measurement of Viable RGCs by Retrograde Labeling with FluoroGold

The experimental procedure was performed as that described in the previous studies^{[16-19]}. Briefly, retrograde labeling of the RGCs was used one week before the rats were euthanized to avoid over counting the RGCs by mixing labeled RGCs with dye engulfing macrophages and microglia. The RGCs of the retinas were counted at a distance of 1 or 3 mm from the center of the optic nerve head to provide the central and mid-peripheral RGC densities, respectively. Five randomly chosen areas of 62,500 μm² each in the central (approximately 40% of the area of the central retina) and mid-peripheral (approximately 30% of the area of the mid-periphery retina) regions of each retina (n=6 per group) were counted by using ImageMaster-pro10 (Amersham Biosciences).

### (6) Flash Visual-Evoked Potential (FVEP)

The experimental procedure was performed as that described in the previous studies^{[16-19]}. Briefly, visual-evoked potentials were recorded 2 weeks after infarction. A visual electrodiagnostic system (Espion, Diagnosys LLC, Littleton, MA, USA) was used to measure FVEP. Based on the definition of first positive going wavelet (P1) in the previous report, the P1 wave was identified and recorded in the FVEP measurement. The latency of the P1 wave and the amplitude of the P1-N2 wave among groups (n=6 rats in each group) were compared for evaluation of visual function.

### (7) Preparation of Optic Nerves

The experimental procedure was performed according to the previous studies^{[16-19]}. Briefly, a segment of the ONs about 5 to 7 mm in length between the optic chiasm and the eyeball was collected upon sacrifice of rats at 4 weeks. The nerves were immediately frozen at −70° C. for histological and IHC study.

### (8) Preparation of Retinal Section

The experimental procedure was performed according to previous studies^{[16-19]}. Briefly, the eyecups, containing the sclera and the retina, were fixed in 4% paraformaldehyde for 2 hours (h) at room temperature. Each retinal cup was cut adjacent to the disc into two pieces. The tissues were dehydrated in 30% sucrose overnight and kept at −20° C. until further processing. A part of the retinal cups was fixed in 4% paraformaldehyde for sectioning.

### (9) In Situ TUNEL Assay for Apoptotic Cell Measurement

The experimental procedure was performed according to the previous studies^{[16-19]}. Briefly, retina frozen sections were stained by TUNEL assay kit (DeadEnd Fluorometric TUNEL System, Promega Corporation, Madison, WI, USA). The TUNEL positive cells in the ganglion cell layer (GCL) of each sample were counted in ten high powered fields (HPF, ×400 magnification).

### (10) IHC of ED1/IBA1 for Measurement of Inflammatory Response

The experimental procedure was performed according to the previous studies^{[16-19]}. ED1 antibodies react against extrinsic macrophages and intrinsic microglia. IBA1 antibody specifically reacts to intrinsic microglia. The ON frozen sections were subjected to IHC of ED1/IBA1. For comparison, the ED1/IBA1 positive cells were counted in six HPFs (×400 magnifications) at the ON lesion site.

### Example 1: Safety Test for Treatment of PEG-GCSF

The safety of intravitreal injection with PEG-GCSF was tested with normal Wistar rats. In this Example, the change of FVEP was measured to evaluate visual function two weeks after intravitreal injection of PEG-GCSF.

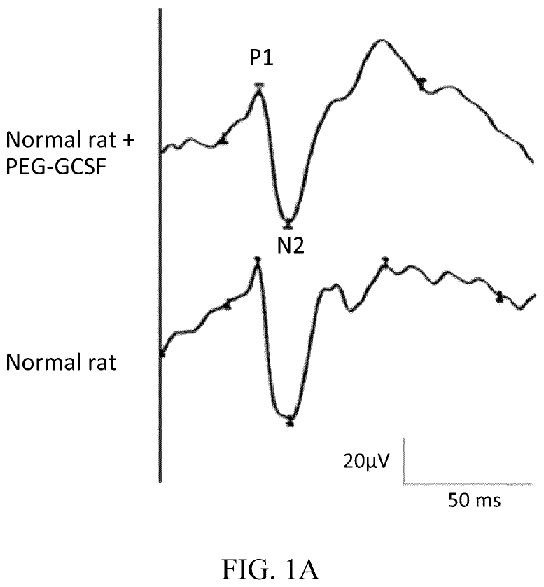

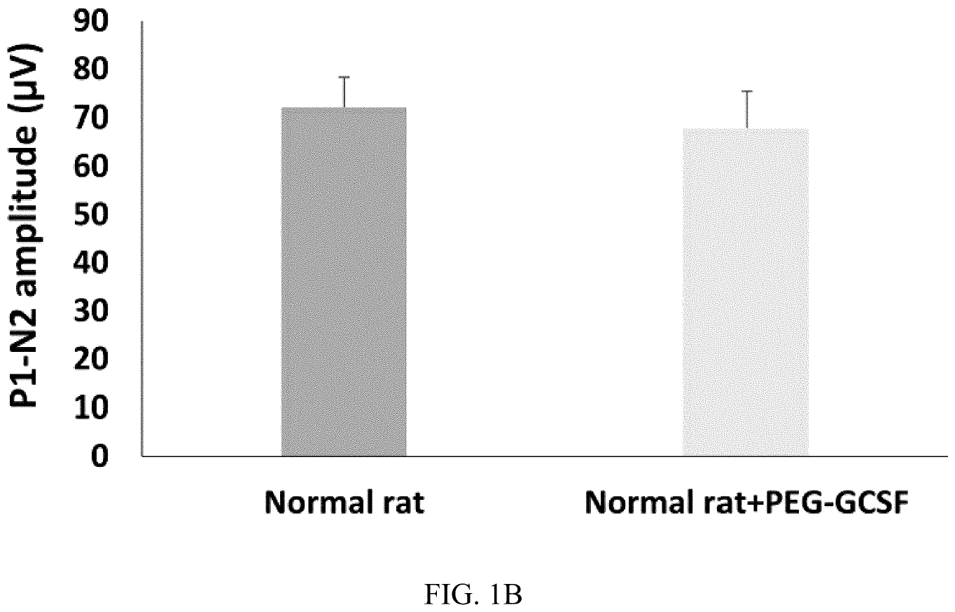

The results were shown in FIG. 1A, indicating that the visual function of rats treated with PEG-GCSF was the same as the control group treated with phosphate buffered saline (PBS). FIG. 1B showed the quantitative data of the amplitude of the P1-N2 shown in FIG. 1A, and it was found that there was no significant difference between both groups. The values of amplitude are expressed as mean±standard deviation (SD) in each group (n=6 rats in each group).

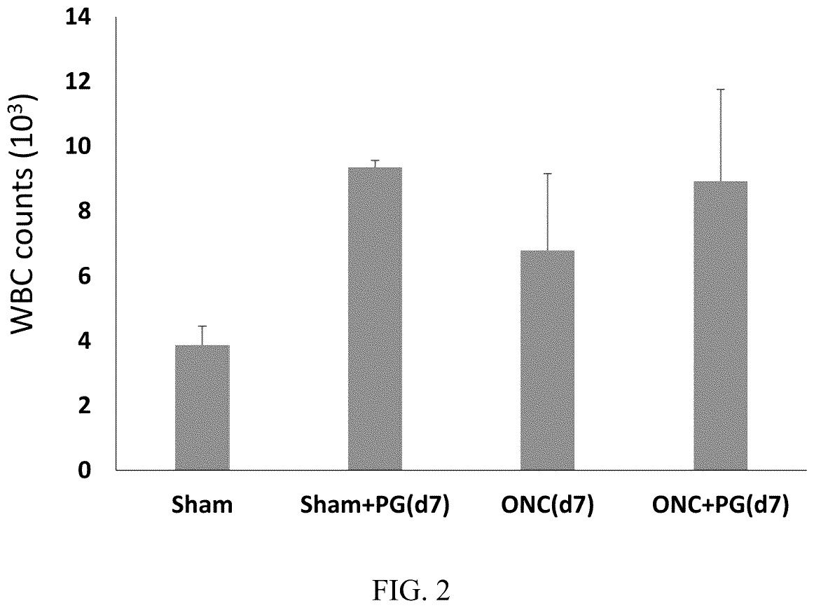

In addition, blood was drawn from cardiac puncture into the heparinized coated tubes by using a 23-gauge needle. All manipulation were performed with animals under general anesthesia. The occurrence of leukocytosis was also determined by counting white blood cells (WBCs) on day 7 post-intravitreal injection with PEG-GCSF in ON crushed rats. Total WBCs were counted by the Cellometer K2 automated cell counter (Nexcelom Bioscience LLC, Lawrence, MA, USA). The results were shown in FIG. 2, and it was found that there was no significant difference between the groups treated with or without PEG-GCSF.

From the above results, it could be seen that the intravitreal injection with PEG-GCSF would neither affect the visual function nor result in leukocytosis, and thus had safety for treating optic neuropathy.

### Example 2: Effect of PEG-GCSF on Visual Function in ONC Rats

In this Example, visual function was assessed by FVEP after treatment with PEG-GCSF via intravitreal injection in the ONC rats. The ONC rats were intravitreally administrated with PEG-GCSF on day 0 post-ONC, and FVEP was measured at 2 weeks post-ONC.

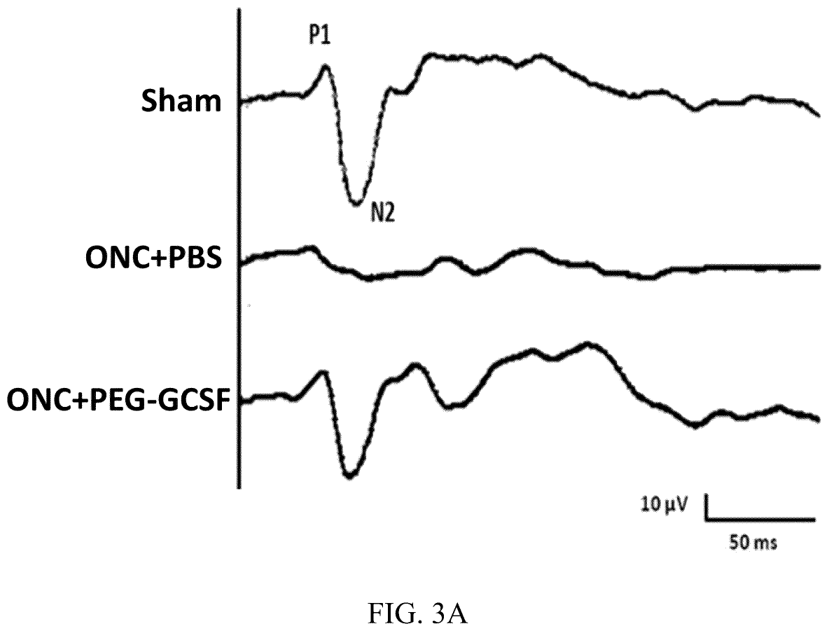

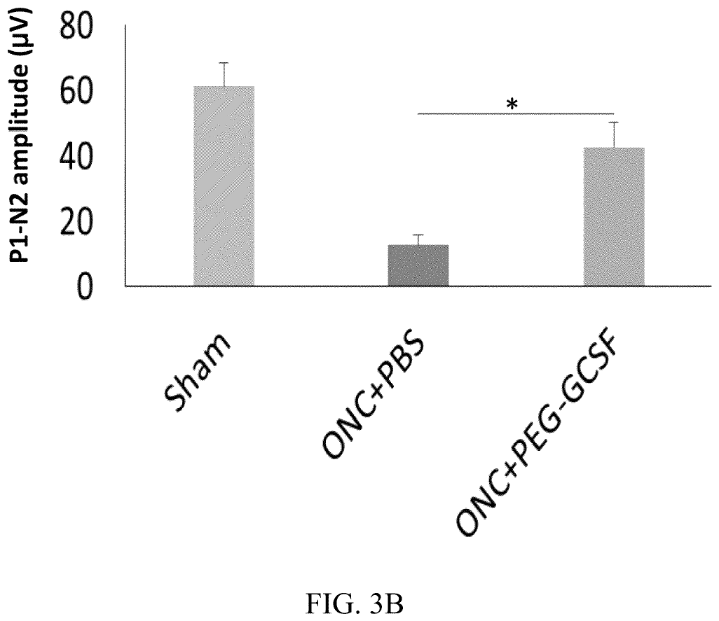

The results were shown in FIGS. 3A and 3B, indicating that intravitreal injection of PEG-GCSF preserved visual function in the ONC model.

### Example 3: Effect of PEG-GCSF on RGC Density in ONC Rats

In this Example, the density of RGC was calculated after treatment with PEG-GCSF via intravitreal injection in the ONC rats. The ONC rats were intravitreally administrated with PEG-GCSF on day 0 post-ONC, and the RGC density was measured at 2 weeks post-ONC.

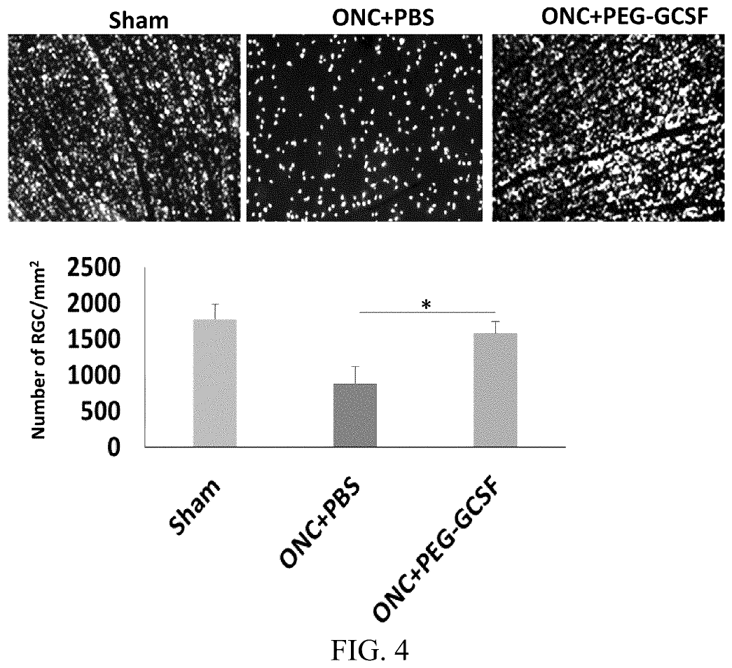

The results were shown in FIG. 4, indicating that intravitreal injection with PEG-GCSF preserved RGC density in the ONC model.

### Example 4: Effect of PEG-GCSF on Inflammatory Response in ONC Rats

In this Example, the effect of PEG-GCSF on inflammatory response was assessed by evaluating inflammatory infiltration and microglia activation in ONC rats post treatment with PEG-GCSF. The ONC rats were intravitreally administrated with PEG-GCSF on day 0 post-ONC, and the immunohistochemistry of ED1 and IBA1 was performed at 2 weeks post-ONC.

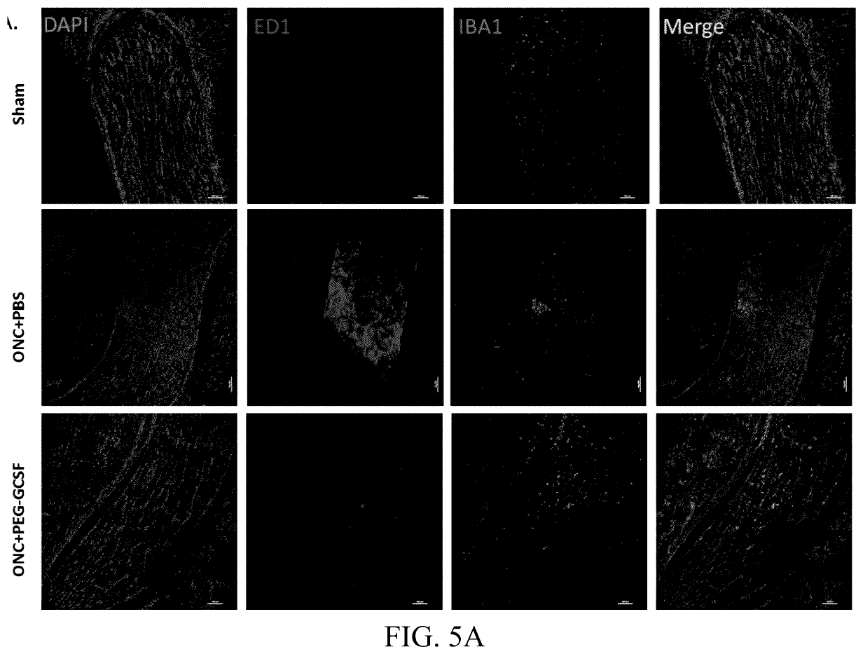

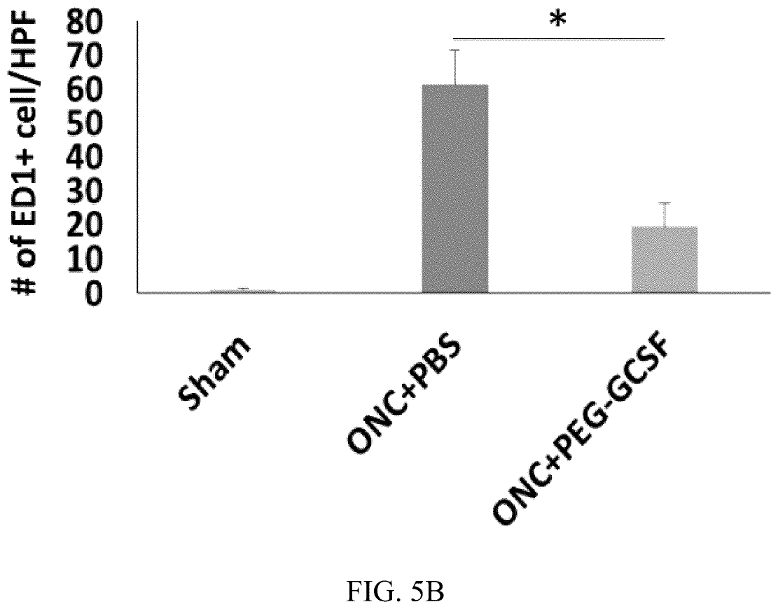

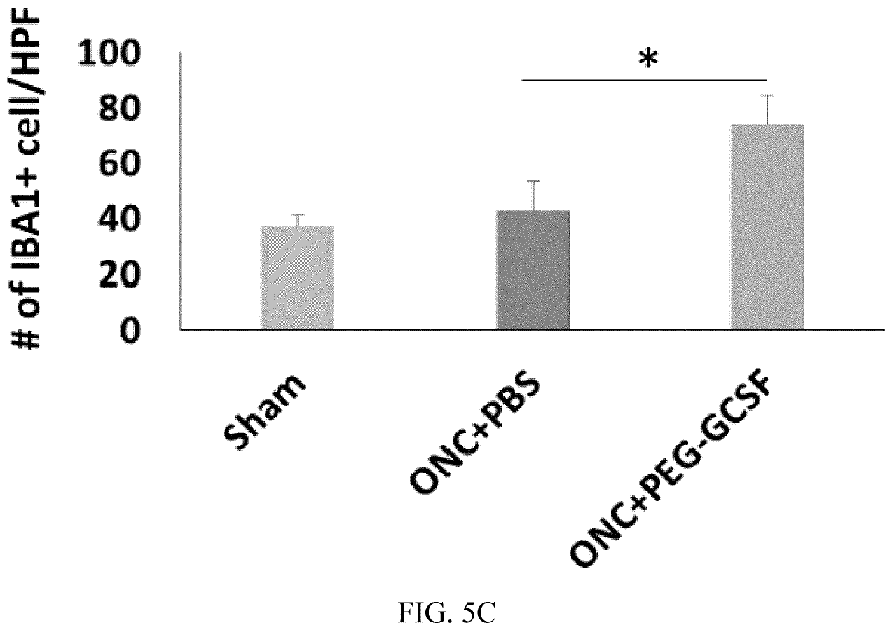

As shown in FIGS. 5A to 5C, by treatment with PEG-GCSF, the ED1 positive cells were decreased, while the IBA1 positive cells were increased at the ON lesion site. It thus could be seen that treatment with PEG-GCSF inhibited macrophage infiltration and induced activation of microglia in ONC rats.

### Example 5: Effect of PEG-GCSF on RGC Apoptosis in ONC Rats

In this Example, RGC death in the RGC layer was assessed by TUNEL assay after treatment with PEG-GCSF via intravitreal injection in the ONC rats. The ONC rats were intravitreally administrated with PEG-GCSF on day 0 post-ONC, and the TUNEL assay was performed at 2 weeks post-ONC.

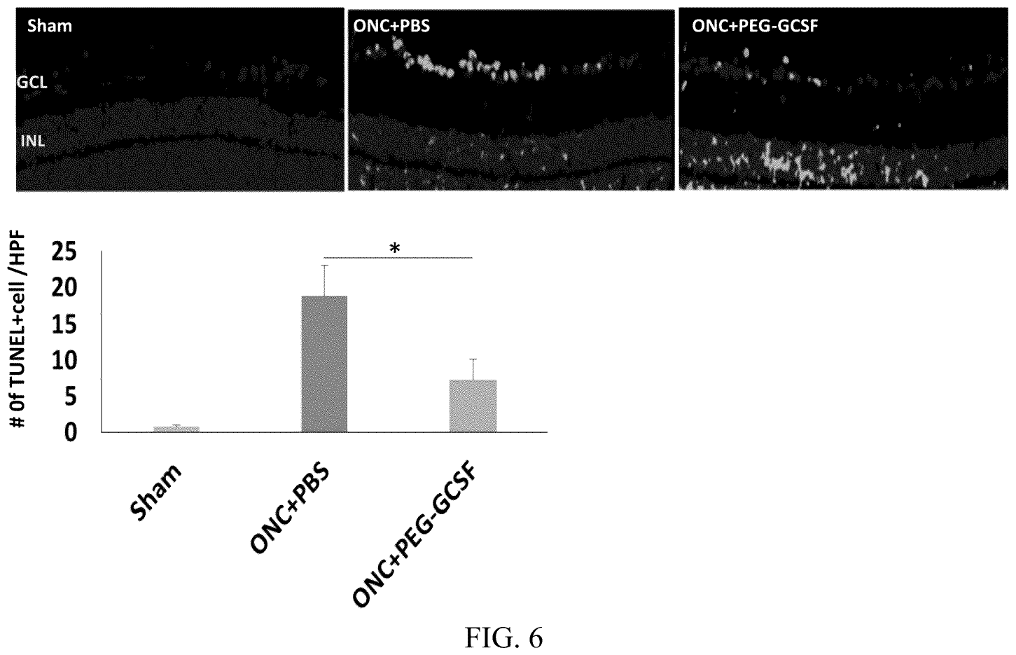

The results were shown in FIG. 6, indicating that there was significant difference between ONC rats treated with or without PEG-GCSF. It thus could be seen that RGC apoptosis was inhibited in ONC animals treated with PEG-GCSF.

### Example 6: Clinical Trial

The clinical trial in this example was a phase 1, semi-experimental trial, which was performed in Hualien Tzu-Chi Hospital. Eight patients were recruited in this study, starting from the 2^{nd} year of project to the 3^{rd} year of project and went through comprehensive eye and systemic examination in the Hualien Tzu-Chi Hospital.

Indirect TON (ITON) patient was defined as reduced best corrected visual acuity (BCVA), visual field, color vision, and positive relatively afferent pupillary defect (RAPD) with normal fundus and optic nerve examination, and no evidence of direct trauma to optic nerves was observed on spiral orbital and optic canal computer tomography (CT) scan. Therefore, all patients had examinations of BCVA, visual field, color vision, RAPD, FVEP, CT scan, and IOP for defining ITON patients one day before PEG-GCSF (Neulasta, Amgen, Inc.) injection. Patients also underwent the renal function test, liver function test, coagulation test, and complete blood count before the treatment. Patients who met the enrollment criteria (inclusion and exclusion, see below) were fully informed of this treatment, and then an informed consent was obtained.

### Inclusion Criteria of TON:

a. 20 to 70 years old;
b. Having indirect traumatic optic neuropathy, one week to 4 weeks after trauma;
c. Normal disc figure and macula appearance;
d. Reduced BCVA (Snellen Chart, less than 20/200) or C-24 central visual field loss more than 10 dB (MD<−10 dB);
e. Color vision defect and positive RAPD;
f. No evidence of direct trauma to ON spiral orbital and optic canal computer tomography (CT) scan;
g. Normal IOP (10 to 21 mm Hg);
h. Normal blood coagulation (prothrombin time (PT): 8 to 12 s; partial thromboplastin time (PTT): 23.9 to 35.5 s; international normalized ratio (INR): 0.85 to 1.15);
i. Adequate hematologic (absolute neutrophil count≥1.5×10⁹/L, hemoglobin≥9 g/dL, platelets≥80×10⁹/L, and PT/PTT/INR≤1.0×upper limit of normal; ULN);
j. Adequate hepatic function (albumin≥2.8 g/dL, serum bilirubin≤2.0 mg/dL or ≤2×ULN, and aspartate aminotransferase and alanine aminotransferase≤5.0×ULN);
k. Adequate renal function (serum blood urea nitrogen (BUN): 6 to 22 mg/dL; serum creatinine: 0.7 to 1.5 mg/dL for men, 0.5 to 1.2 mg/dL for women).

### Exclusion Criteria:

a. Having other injuries that effect on visual function;
b. Direct optic neuropathy;
c. No light perception;
d. Pregnant and breast-feeding women;
e. Having malignancy;
f. Sickle-cell disease;
g. G-CSF allergic reaction;
h. Acute infectious diseases;
i. Benign intracranial hypertension symptoms (1. papilledema in both eyes with no spontaneous venous pulsation; and 2. Increase of peripapillary nerve fiber layer thickness in optical coherence tomography (OCT) imaging);
j. Associated intracranial hemorrhage or severe skull fracture;
k. History or evidence of any other clinically condition that, in the opinion of the investigator, would pose a risk to patient's safety or interfere with study procedures, evaluation, or completion: diabetic retinopathy; maculopathy; uncontrolled hypertension; history of stroke and cardiovascular diseases; and glaucoma.

After patient enrollment, the patient was intravitreally administrated by 0.15 mL of Neulasta (pegfilgrastim) in the injured eye. Firstly, the injured eye was treated with an iodine solution for disinfection and then treated with Alcaine eye drop for topic anesthesia. The 0.15 mL of Neulasta was filled into 1 mL of syringe equipped with 30-gauge beveled needle for intravitreal injection. During injection of Neulasta solution, the anterior chamber decompression for IOP balance was performed. The aqueous humor from anterior chamber was collected for further microarray analysis. After Neulasta treatment, Tobradex eyedrops (Alcon) was given on the injected eye, four times a day. Patient was hospitalized for one day to monitor BCVA, IOP, fundus condition, complete blood count, and any adverse event.

During 3-month follow-up trial, each patient was regularly monitored on day 1, day 7, day 30 and day 90 after treatments by determining the BCVA, the RPAD, the color vision, the visual field, the latency of P-100 wave in FVEP, and the retinal nerve fiber layer (RNFL) thickness, IOP, and complete blood count.

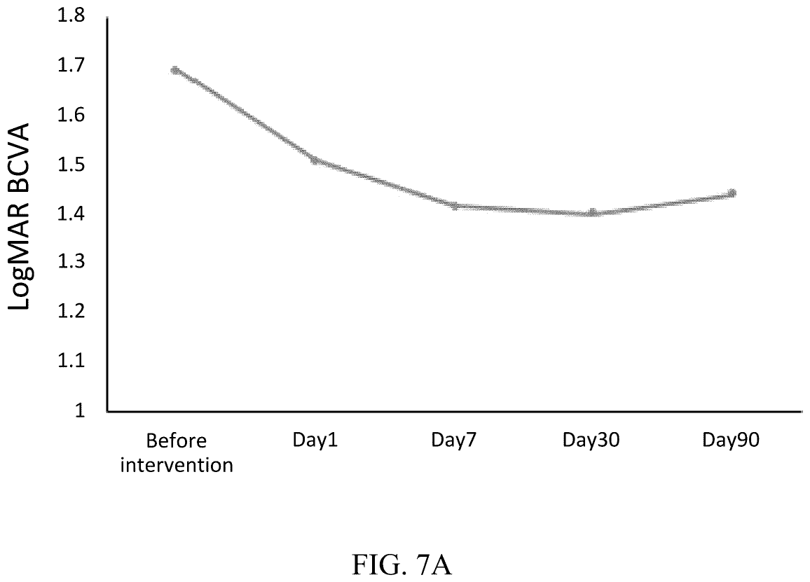

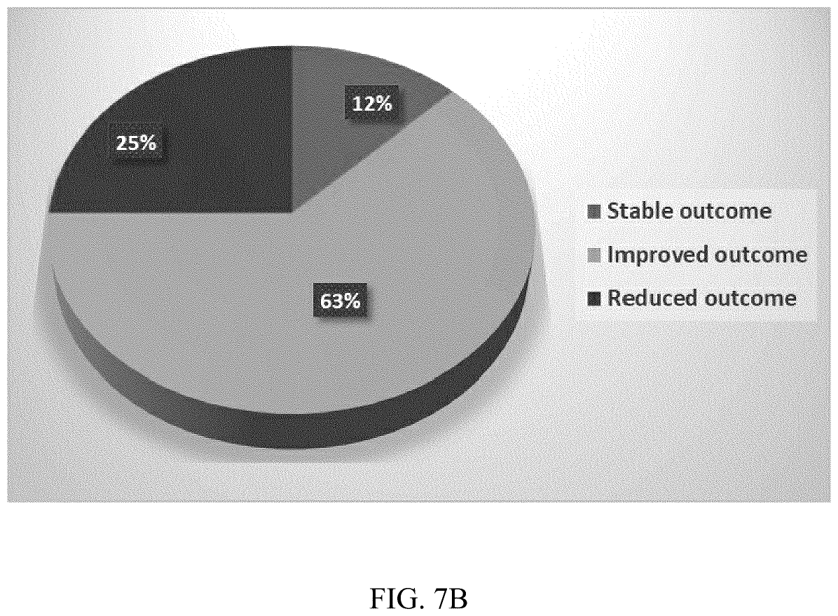

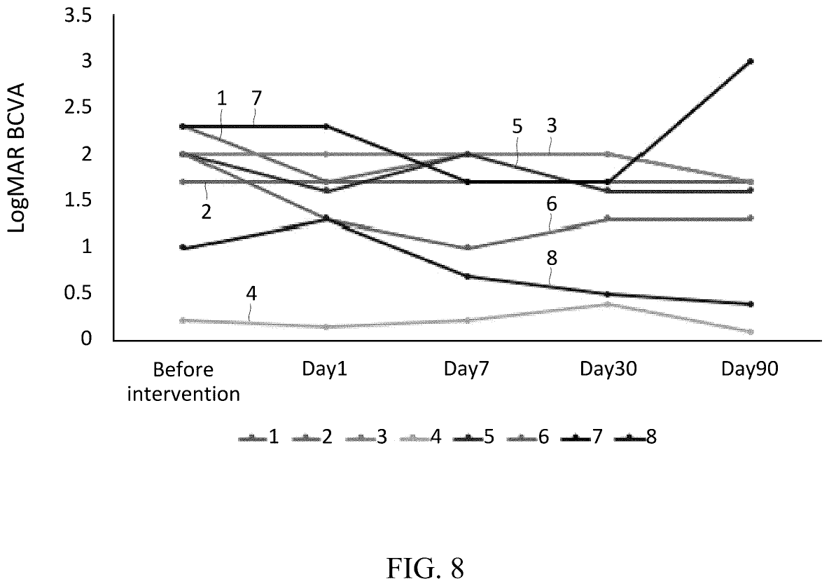

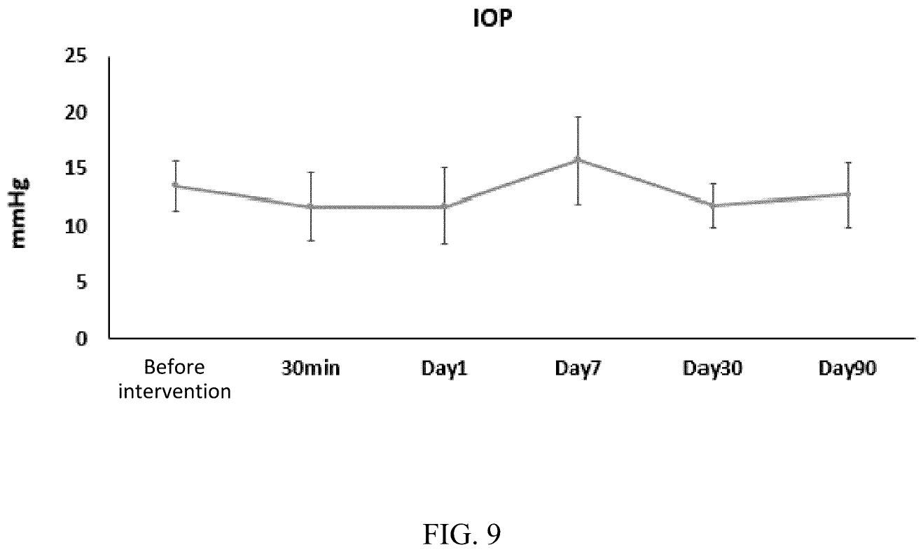

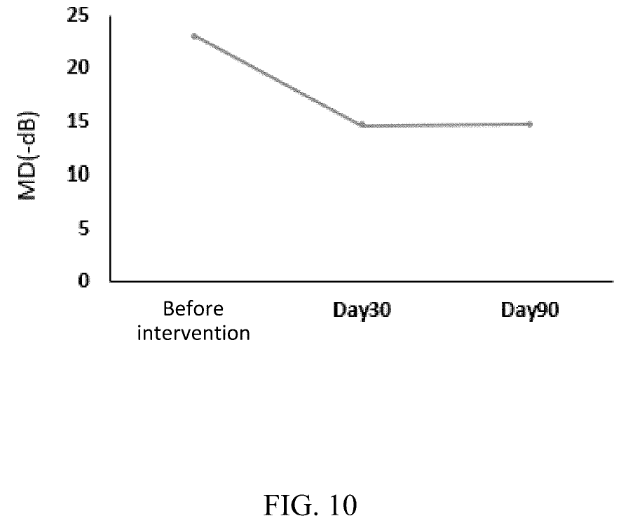

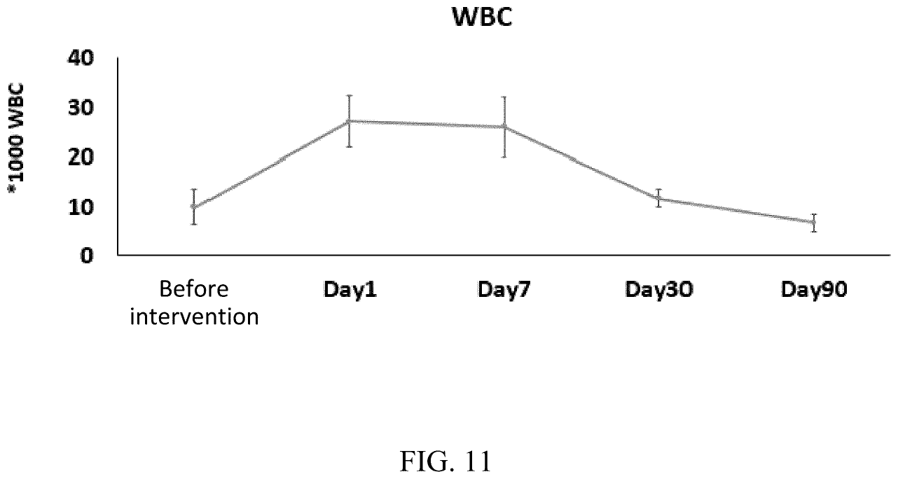

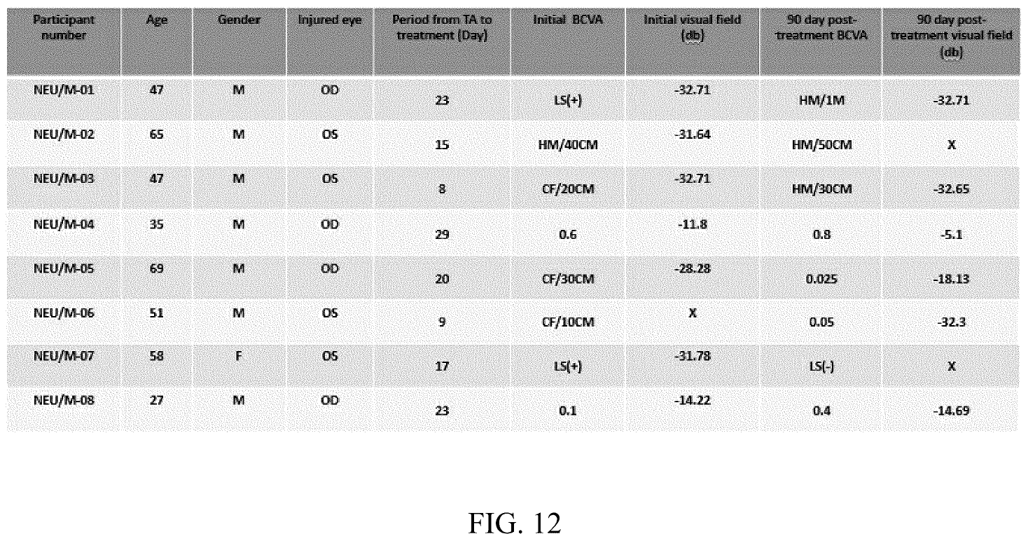

As shown in FIGS. 7A to 7B, the logMAR BCVA of the patients was significantly improved after 30 days from before intervention with the Neulasta treatment. Furthermore, out of the study pool, an improved outcome with the Neulasta treatment has observed in 63% of the patients. As shown in FIG. 8, the logMAR BCVA of the patients was improved after 30 days from before intervention with the Neulasta treatment and more improvement at 90 days from intervention with the Neulasta treatment. As shown in FIG. 12, the initial BCVA was 0.1 and the 90-day post-treatment of BCVA has improved to 0.4 in subjects with traumatic optic neuropathy.

From the above, the results reveal the effects of the administration of the long-acting G-CSF in the treatment of optic neuropathy without adverse effects. For example, single shot of intravitreal injection with PEG-GCSF can exhibit excellent neuroprotective effects in traumatic optic neuropathy.

While some of the embodiments of the present disclosure have been described in detail above, it is, however, possible for those of ordinary skill in the art to make various modifications and changes to the embodiments shown without substantially departing from the teaching and advantages of the present disclosure. Such modifications and changes are encompassed in the scope of the present disclosure as set forth in the appended claims.

All references cited and discussed in this specification are incorporated herein by reference in their entireties and to the same extent as if each reference was individually incorporated by reference.

### REFERENCES

[1] Sarkies N. “Traumatic optic neuropathy.” Eye (London, England) 2004; 18: 1122-1125.
[2] Steinsapir K. D., et al. “Traumatic optic neuropathy.” Survey of Ophthalmology 1994; 38: 487-518.
[3] Anderson R. L., et al. “Optic nerve blindness following blunt forehead trauma.” Ophthalmology 1982; 89: 445-455.
[4] Gross C. E., et al. “Evidence for orbital deformation that may contribute to monocular blindness following minor frontal head trauma.” Journal of neurosurgery. 1981; 55: 963-966.
[5] Levkovitch-Verbin H. “Animal models of optic nerve diseases.” Eye (London, England) 2004; 18: 1066-1074.
[6] Osborne N. N., et al. “Optic nerve and neuroprotection strategies.” Eye (London, England) 2004; 18: 1075-1084.
[7] Lee V., et al. “Surveillance of traumatic optic neuropathy in the UK.” Eye (London, England) 2010; 24: 240-250.
[8] Levin L. A., et al. “The treatment of traumatic optic neuropathy: the International Optic Nerve Trauma Study.” Ophthalmology 1999; 106: 1268-1277.
[9] Mahapatra A. K., et al. “Traumatic optic neuropathy in children: a prospective study.” Pediatric Neurosurgery 1993; 19: 34-39.
[10] Edwards P., et al. “Final results of MRC CRASH, a randomised placebo-controlled trial of intravenous corticosteroid in adults with head injury-outcomes at 6 months.” Lancet (London, England) 2005; 365: 1957-1959.
[11] Singman E. L., et al. “Indirect traumatic optic neuropathy.” Military Medical Research 2016; 3: 2.
[12] Cook M. W., et al. “Traumatic optic neuropathy. A meta-analysis.” Archives of Otolaryngology Head & Neck Surgery 1996; 122: 389-392.
[13] Entezari M., et al. “High-dose intravenous methylprednisolone in recent traumatic optic neuropathy; a randomized double-masked placebo-controlled clinical trial.” Graefe's Archive for Clinical and Experimental Ophthalmology 2007; 245: 1267-1271.
[14] Yu-Wal-Man P., et al. “Surgery for traumatic optic neuropathy.” The Cochrane Database of Systematic Reviews 2005; CD005024.
[15] Steinsapir K. D., et al. “Methylprednisolone exacerbates axonal loss following optic nerve trauma in rats.” Restorative Neurology and Neuroscience 2000; 17: 157-163.
[16] Tsai R. K., et al. “Neuroprotective effects of recombinant human granulocyte colony-stimulating factor (G-CSF) in neurodegeneration after optic nerve crush in rats.” Experimental Eye Research 2008; 87: 242-250.
[17] Huang T. L., et al. “Efficacy of intravitreal injections of triamcinolone acetonide in a rodent model of nonarteritic anterior ischemic optic neuropathy.” Investigative Ophthalmology & Visual Science 2016; 57: 1878-1884.

What is claimed is:

1. A method for treating optic neuropathy in a subject in need thereof, comprising administering to the subject a pharmaceutical composition comprising an effective amount of a long-acting granulocyte-colony stimulating factor (G-CSF) and a pharmaceutically acceptable excipient thereof.

2. The method of claim 1, wherein the long-acting G-CSF is at least one selected from the group consisting of a recombinant G-CSF, a conjugated G-CSF, a G-CSF fusion protein, and any combination thereof.

3. The method of claim 2, wherein the conjugated G-CSF is a non-immunogenic hydrophilic polymer linked to G-CSF.

4. The method of claim 3, wherein the non-immunogenic hydrophilic polymer is covalently linked to the G-CSF.

5. The method of claim 3, wherein the non-immunogenic hydrophilic polymer is at least one selected from the group consisting of polyethylene glycol, polyoxypropylene, polyoxyethylene-polyoxypropylene block copolymer, polyvinylpyrrolidone, polyacyloylmorpholine, polysaccharide, aminocarbamyl polyethylene glycol, and any combination thereof.

6. The method of claim 5, wherein the long-acting G-CSF is a polyethylated G-CSF (PEG-GCSF).

7. The method of claim 2, wherein the G-CSF fusion protein comprises G-CSF fused to a protein selected from the group consisting of albumin and an immunoglobulin fragment of IgG.

8. The method of claim 7, wherein the immunoglobulin fragment is a Fc fragment of the IgG.

9. The method of claim 1, wherein the effective amount of the long-acting G-CSF is in a range of from 10 ng to 100 ng.

10. The method of claim 1, wherein the effective amount of the long-acting G-CSF is in a range of from 11 μg to 2 μg.

11. The method of claim 1, wherein the optic neuropathy is at least one selected from the group consisting of ischemic optic neuropathy, optic neuritis, compressive optic neuropathy, infiltrative optic neuropathy, traumatic optic neuropathy, mitochondrial optic neuropathy, nutritional optic neuropathy, glaucomatous optic neuropathy, toxic optic neuropathy, radiation optic neuropathy, hereditary optic neuropathy, and any combination thereof.

12. The method of claim 1, wherein the pharmaceutical composition is administered to the subject orally, intravitreally, intraperitoneally, intravenously, intradermally, intramuscularly, subcutaneously, or transdermally.

13. The method of claim 12, wherein the pharmaceutical composition is administered to the subject by intravitreal injection.

14. The method of claim 1, wherein the pharmaceutical composition is administered to the subject 1 to 4 times during the treatment.

15. The method of claim 1, wherein the pharmaceutical composition is administered to the subject with one single shot during the treatment.

16. The method of claim 1, wherein the pharmaceutical composition is administered to the subject within one month after the optic neuropathy occurs.

17. The method of claim 1, wherein the subject is a mammal.

18. The method according to claim 17, wherein the mammal is selected from the group consisting of a rodent, a murine, a monkey, a guinea pig, a dog, a cat, a cow, a sheep, a pig, a horse, a rabbit, and a human.
